(19) United States
(12) Patent Application Publication
(10) Pub. No.: US 2011/0288182 A1
(43) Pub. Date: Nov. 24, 2011

(54) HEALTH CHARACTERISTIC DULCE DE LECHE CONFECTION COMPOSITIONS AND FLAVORED MILK

(76) Inventors: Gary Ervin, Naples, FL (US); Erin Ervin, Naples, FL (US)

(21) Appl. No.: 13/110,815

(22) Filed: May 18, 2011

Related U.S. Application Data
(60) Provisional application No. 61/345,957, filed on May 18, 2010.

(51) Int. Cl. A23G 3/36; A23L 1/27; A61K 47/44; A23L 3/3454; A23G 3/50; A23L 1/308; A23L 1/30; A23L 1/221

(52) U.S. Cl. 514/777; 426/660; 426/654; 426/262; 426/650; 426/321

(57) ABSTRACT

The present invention provides for Dulce de Leche confection compositions and Dulce de Leche based flavored milk that has an improved, healthier fat structure, unique antioxidant taste profile, lower sugar content, fiber component, and a 0% or greater supplement component that can add additional dietary benefits. Cholesterol reduction can be accomplished within certain embodiments of this invention. Specific ratios of ingredients within our compositions create a taste, texture, mouth feel, and rich and creamy true Dulce de Leche confection without the negative health ramifications of high milk fat content and sugar content characteristic of traditional Dulce de Leche products. Additionally, the method for preparing these Dulce de Leche confection products and flavored milk are covered.

### RELATED APPLICATIONS

The present application claims priority to U.S. Patent Application Provisional application No. 61/345,957, filed on May 18, 2010, the disclosure of which is incorporated by reference herein in its entirety.

### BACKGROUND OF THE INVENTION

1. Field of the Invention

Embodiments described herein relate generally to ingestible Dulce de Leche confectionery compositions that have improved health characteristics, antioxidant content, less sugar, balance of dietary fats and beneficial dietary fiber that facilitate its use use as ice cream toppings, as fillings for confectionery products, as a layer for a confectionery product or food, or as bite size pieces or chews or molded bars or bite size hard candy pieces or shapes. The Dulce de Leche confection also can be used as a base for making flavored milk. Supplements can be added to the Dulce de Leche confections to target certain health issues such as cholesterol control or triglycerides control that supports prevention of cardiovascular disease.

2. Description of Related Art

Dulce de Leche confections currently available in the marketplace are produced in several ways. First, many Dulce de Leche confections are made as pourable syrup and are used on foods like; ice cream, pancakes, cakes, fruit, and shakes. These are made utilizing milk and sugar and are boiled until the Dulce de Leche flavor profile is created. The result is a very rich and creamy intense sweet milk taste. These types of Dulce de Leche syrups have high moisture content, high sugar content at around 55%, high milk fat content at usually 3 grams to over 8 grams per serving.

Second, many Dulce de Leche confections currently available in the market place are made with less moisture content and these products are more like a spread. This type of Dulce de Leche spreadable confection is used on biscuits, with fruit, over ice cream, on cakes and other foods. These Dulce de Leche confections have a thicker rich and creamy sweet milk taste, texture, and exhibit an improved mouth feel versus the pourable forms of Dulce de Leche. This high sugar content combined with high levels of animal saturated fat milk fat within these compositions has negative health implications Vis a Vis established medical science regarding cardiovascular disease and heart disease and can contribute to diabetes.

Currently there are very limited Dulce de Leche confections in the market place that resemble traditional soft butter caramels. The high moisture content of Dulce de Leche makes it difficult to create Dulce de Leche confections that can be produced in edible bite size pieces or chews. Most of the existing products that claim to be Dulce de Leche confections actually are only Dulce de Leche flavored confections and not real Dulce de Leche.

The art of preparing true Dulce de Leche confections includes cooking milk and sugar until it reduces down into a rich and creamy intense sweet caramelized pudding like composition. The flavor is thermally created and has high moisture content. Dulce de Leche, translated from Spanish as “sweet of milk,” is a popular treat in South and Central American countries. Dulce de Leche is known by different names within Mexico and South America including carjeta (Mexico), arequipe (Columbia), fanquilla (Cuba) and majar blanco (Peru). Dulce de Leche is known to be a water based confection and it is incorporated into many products. Haagen Das Dulce De Leche ice cream, Eddy's Grand Dulce de Leche swirled ice cream, and Breyer's Dulce de Leche ice cream are examples of ice cream utilizing Dulce de Leche within their compositions. The high water content of the Dulce de Leche is compatible with classic ice cream production.

Both the high moisture content and water activity of Dulce de Leche, however, limits the types and quality of products that can be manufactured using it. Dulce de Leche is difficult to combine into foods such as cakes, pastries, cookies, or other types of foods that traditional caramel can be more easily incorporated into because of this high moisture content and water activity of Dulce de Leche. The excessive moisture exhibited by traditional Dulce de Leche makes it difficult to create confectionery products such as molded bars or bite size confection formats. The moisture content causes the Dulce de Leche to lose its shape and thus precludes the use of traditional depositing or open extrusion center forming equipment for making Dulce de Leche confections in the same manner that traditional butter caramels are made.

U.S. Pat. No. 7,229,657 to Woelfel, et al. issued June of 2007, describes the preparation of a Dulce de Leche flavored fat based confection which is said to facilitate coating, depositing, molding. This Dulce de Leche flavored fat based confection indicates that the flavor is thermally created by dispersing a sweeter and dairy protein within a fat to create a confection.

The raw confection is then heated long enough to create the thermal flavor of Dulce de Leche. Woelfel, et al. is not a true Dulce de Leche confection. While a Dulce de Leche flavored confection has wider uses within confectionery and foods compared to current confectionery formats available the product has only the Dulce de Leche flavor and lacks the basic components that, by definition, make a true Dulce de Leche confection that includes milk and sugar reduced down into a rich and creamy intense sweet milk treat. A Dulce de Leche flavored confection would not be able to duplicate the flavor, texture, and mouth feel as a confection based on true Dulce de Leche, as it lacks the milk and sugar aspects of a true Dulce de Leche product.

### SUMMARY OF THE INVENTION

It is a feature of an embodiment to provide an improved taste, improved texture, and improved melt, improved mouth feel, creamier Dulce de Leche confection that has less milk fat, a healthier group of fats, lower sugar content, unique antioxidant flavor profile and addition of dietary fiber. It is another feature of an embodiment to create a Dulce de Leche confection that can have an improved balance of dietary fats including saturated fats, polyunsaturated fats, and monounsaturated fats including beneficial Omega 6 and Omega 3 fatty acids. It is another feature of an embodiment to provide an antioxidant flavor profile that gives the Dulce de Leche a specific unique novel flavor profile that comes from the synergistic combination of coconut oil, vanilla powder, and the traditional Dulce de Leche flavors. The flavor profile thus created has a completely unique taste profile vs. all other Dulce de Leche confections or Dulce de Leche flavored confections currently in the market place.

The present embodiments can create a true Dulce de Leche confection that can be formed into bite size chews or bars or other shapes. The embodiments described herein solve the high moisture content issue through combinations of ingredients that improve the health characteristics of the Dulce de Leche confection, while allowing a viscosity necessary to maintain a bite size soft chew similar to a traditional butter caramel.

The embodiments described herein also can create an improved nutritional characteristic range of Dulce de Leche confections including a pourable syrup, a thicker spreadable confection, a molded bar or bite size chews similar to traditional butter caramel, a bite size hard candy confection, and a base for flavored milk. The embodiments described herein create these Dulce de Leche products with superior taste, texture, mouth feel, and flavor profile while offering improved health characteristics including; less milk fat, healthier fat structure, lower sugar content, dietary fiber, and an antioxidant flavor profile.

There is a need for a Dulce de Leche confection that can be deposited or open extrusion center form manufactured. The embodiments utilize a combination of ingredients that create a synergistic effect on the Dulce de Leche confection, permitting it to be processed into confectionery forms similar to traditional butter or milk based caramels. The embodiments overcome the inadequacies that traditional water-based Dulce de Leche products suffer from and enable the production of confections similar to regular caramel based confections, such as combinations within other confections like chocolate, within nutrition bars, within bakes goods like cakes, biscuits, cookies, etc., and within other foods that utilize a confection component with properties similar to caramel.

In accordance with these and other features of the embodiments, there is provided Dulce de Leche confections that have lower sugar content, preferably on the order of 25% to 75% less sugar, when compared to conventional Dulce de Leche confections. Sugar consumption has been tied to increased risk for cardiovascular disease, risk for diabetes, and risk for obesity. Confectionery products especially Dulce de Leche confections are high in sugar content. Many Dulce de Leche confections have a 55% or greater sugar content. The Dulce de Leche confections described herein can significantly reduce the sugar content, while maintaining and actually improving the taste of the product. The Dulce de Leche confection compositions described herein improve the sensory characteristics of Dulce de Leche and moderate the sweetness so that the flavor and taste profile meet a broader consumer appeal.

It is another feature of an embodiment to create Dulce de Leche confections that have a beneficial dietary fiber component. This dietary fiber component provides a specific improved texture and creaminess as well as positive health attributes. This fiber component alters the Dulce de Leche confection formulations in such a way as to permit the manufacturing of molded confectionery bars and permits the formulation to be deposited into various shapes or extruded into various bite size shapes. Traditional Dulce de Leche confectionery products are extremely difficult to produce in molded confectionery bars or as deposited shapes. The high moisture content of Dulce de Leche causes the deposited or extruded forms to lose shape and the making of extruded shapes or deposited shapes is therefore almost impossible. The embodiments described herein utilize inulin, a natural plant based dietary fiber component. This inulin fiber component reacts with the other ingredients within the Dulce de Leche confection compositions described herein such that final viscosity and structure become suitable for traditional confectionery depositing or extruding processes. Within certain embodiments, a Dulce de Leche confection can be prepared that can have a wide range of uses similar to caramel and caramel flavored confections.

In one embodiment, the Dulce de Leche confection composition has improved taste, texture, melt, creaminess and mouth feel, when compared to conventional Dulce de Leche confections, and has a balance of dietary fats plus beneficial essential fatty acids Omega 6 and Omega 3 ALA's, lower sugar content, antioxidants, and dietary fiber.

In accordance with the features and advantages of the various embodiments described herein, there is provided a Dulce de Leche confection composition comprising: (a) from about 20 to about 90% by weight of a Dulce de Leche component; (b) from about 3 to about 25% by weight of a liquid edible oil component; (c) from about 1 to about 8% by weight of a coconut oil component; (d) from about 1 to about 50% by weight of a fiber component; and (e) from about 10 to about 25% by weight of a reducing sugar. Additional embodiments described herein comprise the addition of one or more of the following optional components: (e) from about 1 to about 8% by weight of a vanilla component; (f) from about 0.001 to about 5% by weight of an emulsifier; (g) a supplement selected from the group consisting of: vitamins, minerals, herbs, botanicals, plant derived supplements, animal derived supplements, therapeutic compounds, and mixtures thereof; and (h) an additional component selected from the group consisting of a preservative component.

In accordance with another feature of embodiments described herein, there is provided a method of making a Dulce de Leche confection composition comprising: (a) mixing in a mixing vessel from about 20 to about 90% by weight of a Dulce de Leche component, from about 3 to about 25% by weight of a liquid edible oil component, from about 1 to about 8% by weight of a coconut oil component, from about 1 to about 50% by weight of a fiber component, and from about 10 to about 25% by weight of a reducing sugar; (b) heating the mixture to a temperature within the range of from about 60 to about 75° C. for a period of time within the range of from about 5 to about 25 minutes to dissolve the vanilla powder and emulsify the liquid edible oil and coconut oil component to form a uniform heated mixture; (c) optionally adding additional components; (d) optionally heating the uniform heated mixture to a temperature providing a slow boil of the components (from 100° C. to about 115° C.) for a period of time within the range of from about 2 to about 25 minutes; and (e) cooling the mixture to provide a Dulce de Leche confection composition.

These and other features of the preferred embodiments will be readily apparent to those having ordinary skill in the art upon reading the detailed description that follows:

### DETAILED DESCRIPTION OF THE INVENTION

All percentages and ratios are calculated by weight unless otherwise indicated. All percentages and ratios are calculated based on the total final composition unless otherwise indicated. Referenced herein may be trade names for components including various ingredients utilized in the present invention. The invention described herein is not intend to be limited by materials under a given trade name. Equivalent materials (e.g., those obtained from a different source under a different name or reference number) to those referenced by trade name may be substituted and utilized in the description.

As used herein “fiber” generally denotes material derived from plant cell walls and that is not digestible by human digestive enzymes, including soluble fiber and insoluble fiber. The fiber component can be naturally derived or synthetic. A portion of the fiber component can be non-starch polysaccharides, including soluble and non soluble fiber. As used herein “soluble fiber” denotes plant gums and oligosaccharides, or modified gums, modified celluloses, non-starch polysaccharides that are soluble in water, some of which can form viscous gels.

The Dulce de Leche confection compositions described herein are based primarily on a true Dulce de Leche component (i.e., a commercially available Dulce de Leche, or one prepared in accordance with well known recipes and techniques), plus nutritive ingredients including an edible oil blend consisting of highly unsaturated fats, a coconut component, a fiber component, a reducing sugar component, and optionally a vanilla powder component that create a Dulce de Leche confection that can be produced in the traditional forms that Dulce de Leche are sold in that include pourable syrup, spreadable thicker confection, and all the traditional formats that butter type or vegetable based fat caramel products can be produced in. The ability to create a Dulce de Leche confection composition that can be molded or extruded into bite size pieces is a significant difference between our invention and the prior art Woelfel patent Dulce de Leche flavored fat based confection. The Woelfel patent disclosure does not refer to any format of product and only identifies a moisture content level of from 5% to 10% which would only offer the properties to produce a Dulce de Leche flavored fat confection in formats similar to traditional vegetable or hydrogenated vegetable fat caramel flavored confections. The Woelfel patent disclosure also has no improved health characteristics, whereas the Dulce de Leche confection compositions described herein have a significantly improved nutritional profile and lower cholesterol when compared to conventional Dulce de Leche confections.

It is a feature of the embodiments described herein to create a Dulce de Leche confection composition that can help control cholesterol and high triglycerides, both of which contribute to increased risk of cardiovascular disease. Cholesterol, and more specifically the plaque that it causes in the bloodstream, and high triglycerides are leading contributing factors to cardiovascular disease and coronary heart disease. In certain embodiments, supplements may be added to the Dulce de Leche confection compositions that include plant sterols and Omega 3 DHA/EPA. These two supplements have proven beneficial effects on cholesterol control and for lowering the risk for developing cardiovascular disease in the human body, as they are known to remove bad cholesterol plaque from the bloodstream. The science behind how plant sterols and Omega 3 DHA/EPA supplements affect cholesterol and triglycerides in the human body is well established. Below are several internet citations that reference scientific evidence confirming the relationship between cholesterol control and triglyceride control using plant sterols and Omega 3 DHA/EPA supplements within the daily diet;

Plant Sterol Medical Science Cholesterol Control:

http://mednews.wustl.edu/tips/page/normal/6744.html

http://www.webmd.com/cholesterol-management/features/low-cholesterol-diet-plant-sterols-stanols

Omega 3 DHA/EPA Medical Science Cholesterol Control:

http://dhaomega3.org/index.php?category=health-conditions&title=Mar08-10

http://circ.ahajournals.org/cgi/content/full/106/21/2747 poundsSEC2

It is another feature of an embodiment to provide a Dulce de Leche confection composition that can support a claim for a nutritional structure that the medical community, including the FDA, would concede is heart healthy. Within certain embodiments, the Dulce de Leche confection compositions in the form of a taffy or a chew can provide superior taste and have a balance of dietary fats, significantly lower sugar content, increased antioxidant content and with significantly lower milk fat content, when compared to conventional Dulce de Leche confection compositions. The Dulce de Leche confection compositions described herein contain the beneficial essential fatty acids Omega 6 linoleic and Omega 3 ALA's alpha-linolenic in the compositions based on the healthier edible oils that are preferably utilized herein. Supplements also can be added to a caramel flavored Dulce de Leche confection composition that delivers therapeutic amounts of fiber, plant sterols, Omega 3 DHA/EPA and other supplements that can target cholesterol control and support lowering the risk for cardiovascular disease. A “heart healthy” claim therefore may be made based on this structure of the Dulce de Leche supplement confection compositions described herein. The internet citation below shows the FDA approval that permitted Frito Lay to market snacks that have substantial amounts of saturated fats replaced with unsaturated fats. Science links the replacement of saturated fats with unsaturated fats as a way to reduce the risk of heart disease and other chronic health problems that face the US and global populations.

http://www.foodnavigator-usa.com/Financial-Industry/FDA-approves-unsaturated-fat-health-claim

The ability to significantly lower milk fat, lower sugar content, provide dietary fiber, increase antioxidants, and provide a healthier balance of dietary fats using the Dulce de Leche confection compositions described herein offers the potential for a profound effect on consumer's health. The preferred embodiments use healthier saturated fat from coconut oil and increase good monounsaturated and polyunsaturated fat content, lower sugar content, deliver antioxidants and dietary fiber to the consumer.

Many patents have been granted for caramel flavored confections and caramel products. For example, Mars Incorporated has a patent granted for a Dulce de Leche flavored fat based confection. The embodiments described herein focus on improving the health characteristics of Dulce de Leche confection compositions and expanding the potential consumer market for such compositions by providing equivalent or superior taste, texture, creaminess, and melt when compared to conventional Dulce de Leche confections. The embodiments also provide a reduction of negative health aspects including milk fat and sugar content that both exhibit negative health characteristics. Sugar is known to be one of the largest contributing factors to health problems. The present embodiments utilize healthier alternative edible oils and fats, plus make use of dietary fiber to displace sugar, thereby providing the Dulce de Leche confection compositions with improved health characteristics. The present embodiments displaces the sugar typically present in Dulce de Leche confection compositions with such alternative edible oils and fats and in certain embodiments can reduce sugar by up to 75%, while providing a healthy group of dietary fats and beneficial dietary fiber. The present inventors have found, however, that simply displacing some of the sugar present in conventional Dulce de Leche confection compositions (by using less of the Dulce de Leche), sometimes results in long-term stability and shelf life issues, including crystallization and the like. While not intending on being bound by any theory of operation, the inventors believe that even more of the Dulce de Leche can be displaced by a reducing sugar, whereby the reducing sugar unexpectedly and/or synergistically provides the requisite stability and reduces the tendency to crystallize.

The Dulce de Leche confection compositions can be made into molded bars or chews, or bite size pieces similar to traditional caramel. Global confectionery manufacturers have found it extremely difficult to nearly impossible to process Dulce de Leche into a format that can be deposited or extruded into molded shapes or bite size chews. The high moisture content typically present in conventional Dulce de Leche confection compositions causes traditional manufactured deposited or extruded compositions to lose their shape. For this reason, it is nearly impossible to manufacture such confections on a large scale. The Dulce de Leche confection compositions described herein, especially the interaction of the dietary fiber component, permits the use of conventional commercial confectionery depositing or extruding processes to create stable structure finished confections. In certain preferred embodiments, the compositions described herein can provide the consumer with beneficial dietary fiber at a supplement level, essential fats, Omega 6, and Omega 3 supplements, when compared to conventional Dulce de Leche compositions that contain no fiber or additional dietary fats.

In accordance with the features and advantages of the various embodiments described herein, there is provided a Dulce de Leche confection composition comprising: (a) from about 20 to about 90% by weight of a Dulce de Leche component; (b) from about 3 to about 25% by weight of a liquid edible oil component; (c) from about 1 to about 8% by weight of a coconut oil component; (d) from about 1 to about 50% by weight of a fiber component; and (e) from about 10 to about 25% by weight of a reducing sugar. Additional embodiments described herein comprise the addition of one or more of the following optional components: (e) from about 1 to about 8% by weight of a vanilla component; (f) from about 0.001 to about 5% by weight of an emulsifier; (g) a supplement selected from the group consisting of: vitamins, minerals, herbs, botanicals, plant derived supplements, animal derived supplements, therapeutic compounds, and mixtures thereof; and (h) an additional component selected from the group consisting of a preservative component.

A particularly preferred Dulce de Leche confection composition, without the addition of a reducing sugar, comprises:

a. a Dulce de Leche component similar to Nestle's La Lechera at 69%

b. liquid edible oil component at 18% (blend of canola, soybean oil and sunflower oil)

c. 3.8% coconut oil component (medium heat refined coconut oil)

d. fiber component at 5.4% (naturally derived Inulin)

e. vanilla component at 3.8% (natural vanilla extract on a dextrose base)

f. emulsifier component at 0.5% (lecithin)

In another preferred embodiment, the Dulce de Leche component similar to Nestle's La Lechera is a pourable Dulce de Leche, preferably one manufactured and sold commercially by Nestle as La Lechera. In another preferred embodiment, some of the Dulce de Leche component is displaced with a reducing sugar, in which preferred embodiment, all of the above ingredients are utilized, except only 54% of the Dulce de Leche is used, and 15% of corn syrup is added. In all of the embodiments described herein, the amount of Dulce de Leche component can be displaced by an equivalent amount of a reducing sugar. For example, the amount of Dulce de Leche component could be 49% by weight and about 20% by weight of reducing sugar would be added to the composition.

Another preferred embodiment includes a composition comprising: a. a Dulce de Leche component similar to Nestle's La Lechera at 69%, by weight; b. liquid edible oil component at 18% (blend of canola, soybean oil and sunflower oil); and c. 3.8% coconut oil component (medium heat refined coconut oil). A further embodiment includes d. a fiber component at 5.4% (naturally derived inulin), by weight of the composition. Yet another preferred embodiment further includes e. a vanilla component at 3.8% (natural vanilla extract on a dextrose base), by weight of the composition. Another preferred embodiment further includes f. an emulsifier component at 0.5% (lecithin), by weight of the composition. Additional embodiments include the further addition of at least about 0% by weight or greater of a supplement selected from the group consisting of: vitamins, minerals, herbs, botanicals, plant derived supplements, animal derived supplements, therapeutic compounds, and mixtures thereof, and optionally one or more additional preservative components.

Dulce De Leche Component

The compositions described herein includes a Dulce de Leche component as the primary ingredient. It is preferred that the Dulce de Leche component be in syrup form similar to Nestle's Dulce de Leche pourable product. Preferred embodiments envision the use of commercially available Nestle's Dulce de Leche pourable products, or a pourable Dulce de Leche product made using techniques known in the art. Skilled artisans will be capable of making a pourable Dulce de Leche component, using the guidelines provided herein, coupled with what is generally known in the art. It is preferred that the Dulce de Leche ingredient has good taste, texture, and mouth feel as well as the rich and creamy intense sweet milk flavor characteristic of a high quality Dulce de Leche product. The Dulce de Leche component preferably is present in an amount of 20% or greater, more preferably 20% to 90%, still more preferably 30% to 80%, still more preferably 40% to 70%, and most preferably 50% to 69%, by weight of the composition. If a reducing sugar is utilized, as is particularly preferred, the amount of Dulce de Leche component can be displaced by the respective amount of reducing sugar (most preferably about 15% by weight, based on the weight of the composition).

Liquid Edible Oil Component

The Dulce de Leche confection compositions described herein preferably include an edible oil component at 3% to 25%, more preferably 8% to 20%, and most preferably 10% 18%, by weight of the composition. The edible oil component makes up part of the group of ingredients that adjust the taste, texture, and improve the melt and creaminess of the Dulce de Leche confection composition . The interaction of the group of highly unsaturated oils (preferably included as at least a part of the liquid edible oil component) with the coconut oil and milk fat component (of the Dulce de Leche component) is believed to create an improved elasticity of the Dulce de Leche confection compositions in the preferred embodiments.

The edible oil component preferably includes monounsaturated and polyunsaturated fats, and preferably includes, for example, a blend of canola, soybean oil, and sunflower oil. Non-limiting examples of an edible oil component acceptable for use in the embodiments preferably has a low saturated fat content and a high unsaturated fat content including monounsaturated and especially polyunsaturated oils. The edible oil component preferably has no specific flavor, and is basically bland or somewhat buttery in taste. The edible oils component can be comprised of one or more of the following; canola oil, soybean oil, safflower oil, sunflower oil, sesame oil, walnut oil, olive oil, flaxseed oil, chia seed oil, almond oil, corn oil, grape seed oil, peanut oil, other nut oils, and synthesized or reorganized oils, and combinations thereof. Use of certain oils, preferably including walnut or chia seed oil, provides the Dulce de Leche confection composition with a structure having an optimal balance between saturated, monounsaturated, and polyunsaturated fats and would include beneficial amounts of Omega 3 ALA's and Omega 6 essential fatty acids.

Coconut Oil Component

The Dulce de Leche confection compositions described herein contain anywhere from 1% to 8% of coconut oil, more preferably 2% to 7%, and most preferably 3% to 6%, by weight of said composition. The coconut oil preferably is a medium refined coconut oil comprised of 0% to 16% virgin coconut oil, and 84% to 100% refined coconut oil, and forms an important part of the flavor profile of the composition. The coconut oil component improves the taste, texture, melt, and creaminess of the compositions. Any medium heat processed coconut oil can be used in the embodiments described herein.

Fiber Component

The Dulce de Leche confection compositions described herein include from about 1% to about 50% of a fiber component, more preferably 2% to 40%, still more preferably 3% to 30%, and most preferably from about 4% to about 25%, of a fiber component, based on the total weight of the composition. The preferred fiber component for use in the embodiments described herein is a naturally derived inulin. Inulin acts as a thickening agent and also improves the texture of the Dulce de Leche confection composition. Specifically, the presence of inulin permits the compositions described herein to de deposited or extruded into molded shapes or bite size pieces. As will be appreciated by those having ordinary skill in the art, conventional Dulce de Leche compositions are nearly impossible to manufacture into molded shapes or bite size pieces because of their high moisture content. The inventors solved this problem by using a combination of components described herein such that the Dulce de Leche confection compositions can be molded in shapes and deposited or extruded and then retain a desired shape.

Various load rates of inulin can be incorporated in the compositions to create improved texture and at certain load rates can provide dietary benefits including promoting a healthy digestion system, controlling blood sugar levels, and providing probiotic benefits.

Inulin is indigestible by human enzymes ptyalin and amylase, whose primary function is the digestion of starch. As a result, inulin passes through much of the digestive system intact. Inulin is a highly effective prebiotic, stimulating the growth of beneficial probiotic bacteria in the gut. Inulin is used in low fat products because of its ability to provide a creamy smooth texture to products. Inulin is a dietary fiber and is believed to activate beneficial bacteria in the digestive tract. The activation of these bacteria is thought to reduce the risk of bowel cancer.

Inulin has a mildly sweet taste, but does not affect blood sugar levels and is recommended for diabetics. Inulin has been clinically proven to increase calcium absorption., and also improves the body's uptake of calcium that comes from the milk portion of the Dulce de Leche confection compositions described herein. People have used plants containing inulin to help relieve diabetes mellitus, a condition characterized by hyperglycemia and/or hyperinsulinemia.

Reducing Sugar Component

The Dulce de Leche confection compositions of the preferred embodiments displace a portion of the Dulce de Leche component with a concomitant amount of a reducing sugar component. The amount of reducing sugar employed preferably ranges from about 10 to about 30% by weight, more preferably from about 12 to about 25% by weight and most preferably about 15% by weight. When utilized, which is particularly preferred herein, the amount of Dulce de Leche component would be decreased by the same weight percentage. Any reducing sugar can be used in the preferred embodiments, including for example, corn syrup, and other oligosaccharides.

Vanilla Powder Component

The Dulce de Leche confection compositions described herein preferably include from about 1% to about 8% of a vanilla powder component, more preferably 2% to 7%, and most preferably 3% to 6%, by weight of the composition. This vanilla powder component preferably is derived from a vanilla extract sprayed on a dextrose base. This vanilla powder component makes up part of the flavor profile that along with the coconut oil and Dulce de Leche flavor profile has a synergistic effect on improving the taste, texture, melt, and creaminess of the Dulce de Leche confection compositions. A particularly preferred vanilla powder component is commercially available from Cook's Flavoring Company. Other similar vanilla powder or natural vanilla extracts also could be used in embodiments described herein.

Emulsifier Component

The Dulce de Leche confection compositions also preferably include from about 0.001% to about 5% emulsifier component, more preferably 0.2% to 4% and most preferably 0.3% to 3%, by weight of the composition. The emulsifier component can be derived from several different ingredients. The present invention preferably uses lecithin as the emulsifier agent. The emulsifier agent acts as an agent to keep the mixture of two or more liquids in which one is dispersed in the other as microscopic or ultramicroscopic droplets. In the Dulce de Leche confection compositions described herein, this includes the water and oil emulsion that creates the smooth texture of the finished product.

In a preferred embodiment, the Dulce de Leche confection composition is in the form of a caramel flavored confection and comprises at least about 0% or greater, by weight of the composition, of a supplement component. The supplement component can include, but is not limited to, vitamins, minerals, herbs, botanicals, plant derived supplements, animal derived supplements, therapeutic compounds, and mixtures thereof.

Non-limiting examples of additional ingredients that may be present in the Dulce de Leche confection compositions described herein include: calcium, potassium, B vitamins, vitamins A, C, D, E, and K, folic acid, other vitamins and minerals commonly known in the art and used for supplementing the diet; extracts and active phytochemicals including ferulic acid (from apples), ginseng, Ginko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine; glucosamine, chondroitin, msm; melatonin, seratonin; and mixtures thereof.

The Dulce de Leche confection compositions in the preferred embodiments may include from about 0% to about 25%, preferably from about 0% to about 10%, and more preferably from about 0% to about 5%, by weight of the composition of a supplement component.

Preservative Components

The Dulce de Leche confection compositions also may include preservatives. Non-limiting examples of suitable preservatives may be selected from the group consisting of: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof. The Dulce de Leche confection compositions preferably include at least about 0% to 2% by weight of a preservative, based on the total weight of the composition.

Method Of Making

In accordance with another feature of embodiments described herein, there is provided a method of making a Dulce de Leche confection composition comprising: (a) mixing in a mixing vessel from about 20 to about 90% by weight of a Dulce de Leche component, from about 3 to about 25% by weight of a liquid edible oil component, from about 1 to about 8% by weight of a coconut oil component, from about 1 to about 50% by weight of a fiber component, and from about 10 to about 25% by weight of a reducing sugar; (b) heating the mixture to a temperature within the range of from about 60 to about 75° C. for a period of time within the range of from about 5 to about 25 minutes to dissolve the vanilla powder and emulsify the liquid edible oil and coconut oil component to form a uniform heated mixture; (c) optionally adding additional components; (d) optionally heating the uniform heated mixture to a temperature providing a slow boil of the components (from 100° C. to about 115° C.) for a period of time within the range of from about 2 to about 25 minutes; and (e) cooling the mixture to provide a Dulce de Leche confection composition.

A preferred method includes mixing a Dulce de Leche component, a liquid edible oil component, a coconut oil component, a fiber component, and a vanilla powder component in a heating vessel; heating the ingredients a temperature within the range of from about 60 to about 75° C. to dissolve the vanilla powder on the dextrose base component and emulsify the liquid edible oil and coconut oil component to form a uniform heated mixture while scraping the vessel during heating to prevent scorching for 10 minutes. If a Dulce de Leche pourable syrup is the desired confection, then the blend would be cooled and tocopherols in an amount of about 0.001% by weight of the composition would be added as a preservative, and the compositions would then be ready to package. On the other hand, if a Dulce de Leche spreadable confection is desired, then after dissolving the vanilla and emulsifying the fats, the heat would be increased to a temperature within the range of from about 100° C. to about 115° C. (preferably about 107° C.) to bring the mixture to a slow boil for around 5 minutes. The length of time that the composition would be boiled determines the viscosity of the final Dulce de Leche spreadable confection composition and once the viscosity is acceptable the blend would be cooled and tocopherols in an amount of about 0.001% by weight of the composition would be added as a preservative, and the composition then would be ready to package.

In certain embodiments, a Dulce de Leche molded confection or deposited or extruded bar or chew is the desired end product. In these embodiments, the length of time the emulsified mixture is boiled would be increased to around 10 minutes or more, the viscosity would be monitored for acceptable results, and this iterative process would continue until the final desired viscosity were achieved. At this point, the composition would again be cooled, and tocopherols in an amount of about 0.001% by weight of the composition would be added as a preservative, and the composition then would be ready to deposit or extrude and package. Additional flavors, colors, or preservatives could be added to the above Dulce de Leche products at the stage that the tocopherols were added. If a Dulce de Leche supplement product is the desired end product the supplement components would also be added at the point that the tocopherols were added into the compositions.

To improve the economics of the above Dulce de Leche manufacturing processes, all the above ingredients including the additional component of a flavor, a color, a preservative, a stabilizing agent, a thickening agent, a supplement, a nutraceutical, a medicine or combinations thereof could be processed together. This could remove the necessity of the second blending and heating stage and would lower the cost of production.

The above processes produce a Dulce de Leche confection composition that has improved taste, texture, melt, and creaminess, when compared to conventional Dulce de Leche confection compositions, and has a balance of dietary fats plus beneficial essential fatty acids Omega 6 and Omega 3 ALA's, antioxidants, and dietary fiber. The Dulce de Leche confection compositions described herein provide an excellent base for flavored milk, which preferably would be processed in a number of ways understood by those skilled in the art of milk processing. Dulce de Leche flavored milk with improved health characteristics would be an important and beneficial addition to the flavored milk category, which is consumed by a wide group of consumers globally.

If a thicker spreadable Dulce de Leche product is the desired end product, or a caramel type of mold bar or bite size chew is required, then the mixture of ingredients should be brought to a slow boil by heating to a temperature within the range of from about 100° C. to about 115° C. (preferably about 107° C.). The mixture should be maintained at this heat for a period of time to achieve the desired viscosity for the finished product desired. The length of time that the composition boils determines the thickness of the final Dulce de Leche confection. For an ice cream topping or fruit dip type of caramel flavored confection, the time would usually be around 5 minutes at a slow boil to create the correct viscosity. If a Dulce de Leche chew or hard candy is the desired end result the length of time for boil would be longer. Those having ordinary skill in the art, using the guidelines provided herein, can determine the appropriate amount of time to boil the mixture to achieve the appropriate viscosity, depending the components used and the final end product desired.

The preferred embodiments described herein are described in more detail in the non-limiting examples that follow. It will be understood and appreciated that a reducing sugar can be utilized in any of the examples to displace a portion of the Dulce de Leche component. It is particularly preferred to utilize about 15% by weight of a reducing sugar, which results in a concomitant decrease in the Dulce de Leche component of 15% by weight. The below examples are included for illustrative purposes only and are not intended to in any way limit the scope of the present invention.

### EXAMPLES

### Example 1

This example provides a pourable Dulce de Leche confection with improved health characteristics, less sugar content, balance of dietary fats, and beneficial antioxidants and dietary fiber suitable for an ice cream topping, fruit dip, pancakes, biscuits, etc., and a spreadable Dulce de Leche confection composition.

68 pounds of Nestle Dulce de Leche pourable syrup, 18 pounds of Crisco Vegetable Oil Blend (Canola, Soybean, and Sunflower), 3.8 pounds of coconut oil medium heat refined, 5.4 pounds of Inulin fiber, 3.8 pounds of vanilla powder supplied by Cooks Flavor Company, and 0.5 pounds of lecithin were added to a kettle equipped with a gas heater and mixer. The ingredients were mixed and then heated to a temperature of 150 to 160° F. over a low flame. The side of the kettle was constantly scraped to prevent scorching, and the mixture was continuously mixed and scraped for 10 minutes to dissolve the sugar and emulsify the fat. The mixture then was cooled and tocopherols at 0.001% by weight of the composition was added to the mixture as a natural preservative to produce a final ice cream topping product.

To produce a spreadable Dulce de Leche confection composition, instead of cooling, the temperature of the mixture was increased to initiate a slow boil. The mixture was mixed and scraped continuously to prevent scorching for about 3 minutes. The viscosity of the batch was then examined, and if sufficient viscosity were achieved, the mixture was cooled and the finished product was a spreadable Dulce de Leche confection composition.

The example described above could be scaled-up to a large scale production, and the products could be placed into either a jar and capped, extruded into a squeezable type of packaging known in the ice cream toppings industry, or extruded in tubs or containers used for spreadable types of Dulce de Leche products.

### Example 2

This example provides a Dulce de Leche chew or hard candy having improved health characteristics, balance of dietary fats, beneficial antioxidants, lower sugar content and dietary fiber.

54.4 pounds of Nestle Dulce de Leche pourable syrup, 14.4 pounds of Crisco Vegetable Oil Blend (Canola, Soybean, and Sunflower), 3 pounds of coconut oil medium heat refined, 20 pounds of inulin fiber, 3 pounds of vanilla powder supplied by Cooks Flavor Company, and 3 pounds of lecithin were added to a kettle as in Example 1, mixed, and heated to a temperature within the range of from about 150 to about 160° F. over a low flame. The side of the kettle was constantly scraped to prevent scorching, and mixing and scraping were continued for 10 minutes to dissolve the sugar and emulsify the fat. The mixture then was brought to a slow boil by increasing the heat gas flame and the ingredients to a temperature of about 225° F. The emulsified mixture was boiled for about 5 to 10 minutes to develop sufficient thickening, all the while continuing to mix the ingredients and scrape the sides of the kettle.

The mixture then was examined for thickness and was either taken off the flame and cooled or boiled for additional time to create the required viscosity for the Dulce de Leche confection chew. When the mixture achieved the desirable thickness, the mixture was cooled to about 93° F., and a preservative component of tocopherols was added in an amount of 0.001% by weight of the composition. The serving size was approximately 36 grams, and there were multiple chews per serving. A hard candy Dulce de Leche confection also was produced by reducing the thickness down and adding additional inulin to create a viscosity that produced a hard candy at room temperature. The serving size also was about 36 grams, and there were multiple hard candies per serving.

### Example 3

This example produces a Dulce de Leche chew or hard candy with improved health characteristics, balance of dietary fats, beneficial antioxidants and dietary fiber plus plant sterols and Omega 3 EPA/DHA supplements.

54.4 pounds of Nestle Dulce de Leche pourable syrup, 14.4 pounds of Crisco Vegetable Oil Blend (Canola, Soybean, and Sunflower), 3 pounds of coconut oil medium heat refined, 20 pounds of inulin fiber, 3 pounds of vanilla powder supplied by Cooks Flavor Company, and. 3 pounds of lecithin were added to a kettle, mixed, heated, and scraped as described above in Example 2. The temperature of the mixture then was increased to bring the mixture to a slow boil as described above in Example 2, and the mixture was boiled for about 5 to 10 minutes to develop sufficient thickening (continue mixing and scraping). As described in Example 2, above, the mixture was examined for the appropriate thickness, and once the desired thickness were achieved, the mixture was cooled to about 93° F., and plant sterols were blended into the mixture in an amount of about 0.8 grams per serving. A stabilized Omega 3 DHA/EPA also was added in an amount of about 260 mg per serving to provide a caramel flavored confection composition. Then, a preservative component of tocopherols were added at 0.001% of the composition. The serving size was about 14 grams per chew.

A hard candy caramel flavored confection also was prepared as described in Example 2 above, by reducing the thickness by the addition of inulin to provide a viscosity that would produce a hard candy at room temperature. The mixture was cooled, plant sterols were added in an amount of 0.8 grams per serving, and a stabilized Omega 3 DHA/EPA was blended in at 260 mg per serving. The serving size also was about 14 grams per hard candy.

All documents referred to herein are, in relevant part, incorporated by reference in their entirety; the citation of any document is not to be construed as an admission that it is prior art with respect to the present invention. To the extent that any meaning or definition of a term utilized herein conflicts with any meaning or definition of the term in a document incorporated by reference, the meaning or definition assigned to the term herein shall govern.

While particular embodiments of the present invention have been illustrated and described, it will be readily apparent to those skilled in the art that various changes and modifications can be made to the preferred embodiments without departing from the spirit and scope of the invention.

What is claimed is:

1. A Dulce de Leche confection composition comprising:
(a) from about 20 to about 90% by weight of a Dulce de Leche component;
(b) from about 3 to about 25% by weight of a liquid edible oil component;
(c) from about 1 to about 8% by weight of a coconut oil component;
(d) from about 1 to about 50% by weight of a dietary fiber component; and
(e) from about 10 to about 25% by weight of a reducing sugar.

2. The composition of claim 1, further comprising:
(f) from about 1 to about 8% by weight of a vanilla powder component; and
(g) from about 0.001 to about 5% by weight of an emulsifier component.

3. The composition of claim 1, wherein the Dulce de Leche component is present in an amount of from about 50 to about 70% by weight, based on the total weight of the composition.

4. The composition of claim 1, wherein the edible oil component is present in an amount of from about 4 to about 20% by weight, based on the total weight of the composition.

5. The composition of claim 4, where the edible oil component is comprised of the one or more oils selected from the group consisting of canola oil, soybean oil, safflower oil, sunflower oil, sesame oil, walnut oil, olive oil, flaxseed oil, chia seed oil, almond oil, corn oil, grape seed oil, peanut oil, other nut oils, and mixtures thereof.

6. The composition of claim 4, wherein the edible oil is comprised of a blend selected from a blend of canola, soybean oil, and sunflower oil, and a blend of canola, soybean oil, and olive oil.

7. The composition of claim 1, wherein the coconut oil component is a blend comprising from about 0% to 16% virgin coconut oil, and from 84% to 100% refined coconut oil.

8. The composition of claim 1, wherein the coconut oil component is present in an amount of from about 3 to about 5% by weight, based on the total weight of the composition.

9. The composition of claim 1, wherein the dietary fiber component is present in an amount of from about 5 to about 25% by weight, based on the total weight of the composition.

10. The composition of claim 9, wherein the fiber component is selected from one or more of the group consisting of naturally derived soluble fiber, naturally derived inulin, inulin extract, synthetic inulin, fructooligosaccharides, galacto-oligosaccharides, xylooligosaccharides, oligo derivatives of starch; husks; brans; psyllium; polysaccharides, starches; polycarbophil; lignin;
arabinogalactans; chitosans; oat fiber; soluble corn fiber, non-digestible corn dextrin; non-digestible wheat dextrin; locust bean gum, derivatives of locust bean gum; hydroxypropylmethyl cellulose (HPMC); pectin; and mixtures thereof.

11. The composition of claim 9, wherein the fiber component is naturally derived inulin.

12. The composition of claim 1, wherein the reducing sugar is present in an amount of about 15% by weight, based on the total weight of the composition.

13. The composition of claim 12, wherein the reducing sugar is corn syrup.

14. The composition of claim 2, wherein the emulsifier component is selected from one or more of the group consisting of lecithin, mono-and diglycerides, modified corn starch, and mixtures thereof.

15. The composition of claim 1, further comprising a supplement component selected from Omega 3 DHA/EPA and plant sterols.

16. A Dulce de Leche confection composition comprising:
(a) about 54% by weight of a Dulce de Leche component;
(b) from 11 to 20% by weight of a liquid edible oil component comprising a blend of canola, soybean oil and sunflower oil;
(c) from 3 to 4% by weight medium refined coconut oil;
(d) from 3 to 20% by weight of inulin fiber;
(e) about 15% by weight of corn syrup;
(f) from 3 to 4% by weight of a vanilla component; and
(g) from 0.001 to 5% by weight of an emulsifier component.

17. A method of making a Dulce de Leche confection composition comprising: (a) mixing in a mixing vessel from about 20 to about 90% by weight of a Dulce de Leche component, from about 3 to about 25% by weight of a liquid edible oil component, from about 1 to about 8% by weight of a coconut oil component, from about 1 to about 50% by weight of a fiber component, and from about 10 to about 25% by weight of a reducing sugar; (b) heating the mixture to a temperature within the range of from about 60 to about 75° C. for a period of time within the range of from about 5 to about 25 minutes to dissolve the vanilla powder and emulsify the liquid edible oil and coconut oil component to form a uniform heated mixture; (c) optionally adding additional components; (d) optionally heating the uniform heated mixture to a temperature within the range of from about 100° C. to about 115° C. for a period of time within the range of from about 2 to about 25 minutes; and (e) cooling the mixture to provide a Dulce de Leche confection composition.

18. The method of claim 17, wherein additional components are added and are selected from the group consisting of a flavor component, a color component, a preservative component, a stabilizing component, an emulsifier component, a thickening component, a supplement component, a nutraceutical component, a medicine component, and mixtures thereof.

19. The method of claim 17 further comprising forming the composition into bite size chews, bite size hard candy pieces or squares.
